(19) United States
(12) Patent Application Publication
(10) Pub. No.: US 2025/0378954 A1
(43) Pub. Date: Dec. 11, 2025

(54) SYSTEM FOR VISUALIZING AND SUPPORTING A CONTEXTUAL DIAGNOSTIC DECISION FOR CONTAGIOUS DISEASES

(71) Applicant: Quidel Corporation, San Diego, CA (US)

(72) Inventors: John Zacharia, San Diego, CA (US); William J. Ferenczy, La Jolla, CA (US)

(21) Appl. No.: 18/878,535

(22) PCT Filed: Jun. 21, 2023
(86) PCT No.: PCT/US2023/068838

§ 371 Date: Dec. 23, 2024

Related U.S. Application Data
(60) Provisional application No. 63/354,587, filed on Jun. 22, 2022.

(51) Int. Cl. G16H 50/20; G16H 10/40; G16H 50/80

(52) U.S. Cl. CPC G16H 50/20; G16H 10/40; G16H 50/80

(57) ABSTRACT

A method for visualizing and supporting a contextual diagnostic decision for contagious diseases is provided. The method includes receiving, from a diagnostic instrument, information regarding a sample cartridge, including a location data and a risk factor, the sample cartridge including multiple test assays for diagnosing multiple infectious diseases. The method also includes selecting a test assay for reporting a diagnostic result, instructing the diagnostic instrument to run the test assay from the sample cartridge, receiving, from the diagnostic instrument, a first data set when the test assay is completed, and assessing a diagnostic result based on the first data set. A system and a non-transitory, computer-readable medium storing instructions to cause the system to perform the above method are also provided.

### CROSS-REFERENCE TO RELATED APPLICATIONS

This application claims the benefit of U.S. Provisional Application No. 63/354,587, filed Jun. 22, 2022, incorporated herein by reference in its entirety.

### TECHNICAL FIELD

The present disclosure is directed to networked systems for clinical diagnostics, surveillance, data analysis, and reporting to health organizations. The systems described herein automate the process of generating a database containing current clinical diagnostic data, analyzing and reporting prior clinical diagnostic results to concerned organizations and agencies in a timely manner, and using the diagnostic results and other location-based information for context in new diagnostics for a contagious disease.

### BACKGROUND

As the number of clinical diagnostic tests increases as well as the number of patients undergoing such tests, the task of collecting and storing the resultant data has increasing importance and challenges. Technical challenges include storing the data for current and later use, ensuring accessibility and management by pertinent parties, and ensuring patient privacy and data security. In addition to diagnostic device networks, current developments of internet resources provide a large amount of geolocation data that may be relevant for disease diagnostics. In addition to data collection, gathering, analysis, and access authorization, there is an unexplored potential for use of prior diagnostic data and other geolocated information for context in the diagnosis of infectious diseases as the diagnostic device network performs new tests in different locations.

Accordingly, there is a desire for privacy-protected and safe collection, maintenance, transmission, analysis, and use of clinically relevant data to provide context for new diagnostics of an infectious disease over different geographical areas. Preferably, these tasks involve minimal human intervention.

### BRIEF SUMMARY

In a first embodiment, a computer-implemented method includes receiving, from a diagnostic instrument, information regarding a sample cartridge to be used on a first subject, the information including a location data and a subject symptom, the sample cartridge including multiple test assays for diagnosing multiple infectious diseases. The computer-implemented method includes selecting, based on the location data and the subject symptom, a test assay for reporting a diagnostic result, instructing the diagnostic instrument to run the test assay from the sample cartridge, receiving, from the diagnostic instrument, a first data set when the test assay is completed, and assessing a diagnostic result based on the first data set.

In a second embodiment, a computer-implemented method includes providing, to a remote server, information regarding a sample cartridge to be used for a diagnostic test on a first subject, the information including a location data and a subject symptom, the sample cartridge including multiple test assays for diagnosing multiple infectious diseases. The computer-implemented method also includes receiving, from the remote server, based on the location data and the subject symptom, a first test assay selected for reporting a diagnostic result, causing a diagnostic instrument to run the first test assay from the sample cartridge, and transmitting, to the remote server, a first data set when the first test assay is completed.

In a third embodiment, a device includes a memory storing multiple instructions, a communications module configured to communicate with a remote server, and a processor configured to execute the instructions to cause the device to perform operations. The operations include to receive, from the remote server, an indication of a test assay for diagnosing an infectious disease to be selected for a subject, the indication based on at least one of a geolocated data indicative of a location of a diagnostic instrument and a prior diagnostic obtained with a one or more diagnostic instruments communicatively coupled with the remote server. The operations also include associating the test assay with multiple values to generate a data set diagnostic, the data set diagnostic stored within a memory of the diagnostic instrument, the multiple values related to one or more of: a test assay identifier, a test assay result, a patient identifier, and a diagnostic instrument identifier. The operations also include, in an embodiment, transmitting the data set diagnostic to the remote server for storage, wherein the remote server generates a report based on the data set diagnostic from each of the one or more diagnostic instruments, the report configured for transmission to a database housed on a database display on a second server or on an end-user workstation.

### BRIEF DESCRIPTION OF THE DRAWINGS

FIG. 1 illustrates an architecture of a system for visualizing and supporting a contextual diagnostic decision for contagious diseases, according to some embodiments.

FIG. 2 is a block diagram illustrating a client device and a server in the architecture of FIG. 1, according to some embodiments.

FIG. 3 illustrates a feedback based on geolocated information associated with a contagious disease in a network, according to some embodiments.

FIGS. 4A-4D illustrate different screenshots from a webpage hosted by a server in a system for visualizing and supporting a contextual diagnostic decision for contagious diseases, according to some embodiments.

FIG. 5 illustrates a bar chart indicative of test types performed in a network of diagnostic instruments, according to some embodiments.

FIG. 6 illustrates a table including data for multiple diagnostic tests, patients, and locations in a network of diagnostic instruments, according to some embodiments.

FIG. 7 illustrates a map including a sequence indicative of the spread of an infectious disease over different portions within a large geographical area, for a selected span of time, according to some embodiments.

FIGS. 8A-8B are maps retrieved from a webpage hosted by a server in a system for visualizing and supporting a contextual diagnostic decision for contagious diseases, according to some embodiments.

FIGS. 9A-9C are screenshots of an application installed in a diagnostic device including messages to the user, according to some embodiments.

FIG. 10 is a flow chart illustrating steps in a method for visualizing and supporting a contextual diagnostic decision for contagious diseases, according to some embodiments.

FIG. 11 is a flow chart illustrating steps in a method for performing a diagnostic test for an infectious disease, according to some embodiments.

FIG. 12 is a flow chart illustrating steps in a method for collecting data to power an insight engine and decision support tool, according to some embodiments.

FIG. 13 is a block diagram illustrating an example computer system with which the client and network device of FIG. 1 and the methods of FIGS. 10-12 can be implemented.

In the figures, elements and steps having the same or similar reference numerals are associated with the same or similar features or procedures, unless explicitly stated otherwise.

### DETAILED DESCRIPTION

In the following detailed description, numerous specific details are set forth to provide a full understanding of the present disclosure. It will be apparent, however, to one ordinarily skilled in the art, that embodiments of the present disclosure may be practiced without some of these specific details. In other instances, well-known structures and techniques have not been shown in detail so as not to obscure the disclosure.

Infectious diseases include rapidly changing and localized scenarios for clinicians to track, interpret, and adjust their healthcare practice. Current practice is to utilize clinician training and experience, as well as out of clinic contemporary knowledge, to identify probable trends and scenarios. This approach fails when an infectious disease is (1) fast moving, (2) varies from regional trends due to local outbreaks, (3) impacting underserved or less represented populations like transient groups or the elderly, and/or (4) less differentiated in symptoms, such as when a clinical presentation overlaps with or mimics other diseases (such as COVID and influenza).

Typically, clinicians are tasked to evaluate the patient symptoms, propose the right diagnostic inputs, capture and ingest them, and make the right clinical recommendation in a way that requires a significant number of inputs beyond those generated by the patient. This raises the liability for the clinician and/or their organization and raises the likelihood of catastrophic results resulting from human error.

As an example, one of the more challenging scenarios clinicians face is diagnosing undifferentiated patients presenting symptoms that often mirror a generalized immune response, typically known as ‘influenza like illness’ (ILI). Symptoms of ILI may include fever greater than about 100° F., cough or sore throat, and symptoms such as malaise, body aches, headache, loss of appetite, and nausea. These characteristic symptoms present nearly identically across many underlying pathologies, including influenza, COVID-19, RSV, strep, and other viral or bacterial pathogens. However, influenza only causes 35-45% of ILI cases during peak seasons, and many other viral infections can present as flu-like. ILI is of particular concern among elderly people living in old age homes, as a potential cause of epidemic outbreaks, and a common cause for hospitalizations. In some situations, West Nile virus and other retrovirus infections may begin as a febrile ILI. SARS, MERS and other fungal infection may lead to, or start from, ILIs. And ILIs have been associated with serious infectious diseases in mammals such as pigs, horses, cattle, and livestock in general.

Thus, clinicians may be challenged to leverage their experience and identify differentiating features, compare against a local infectious disease situation, recommend further testing or diagnostics, diagnose the patient, and propose a care pathway.

In some cases, tests may be inappropriately prescribed for diagnosis without knowledge of the prevalence of an infectious disease in a nearby location. Many diagnostic tests have a predictive power that results in a significant number of false positives or negatives when the test is not appropriately applied at a population level. For example, consider a population of 2,000 people and a diagnostic test that is 90% sensitive and specific. The positive and negative predictive values of the test vary when the prevalence of the disease is varied:
(A) When the prevalence of the disease is 0%, the test will generate 10 false positives out of every 100 patients tested.
(B) When the prevalence of the disease is 100%, the test will miss 10 infected patients out of every 100 patients carrying the disease.

A clinician that is unaware of local infectious disease trends may inaccurately diagnose a patient as positive or negative based off the wrong test or erroneous test result, resulting in lack of treatment or inappropriate treatment. In addition, lack of differentiating features may require multiple rounds of testing, which can result in a patient waiting days for the right intervention. For certain antiviral options, this can remove a patient from the window of efficacy and limit their treatment options.

Current developments in network technologies enable having a large number of medical and non-medical devices and sensors spread over a large geographic area, providing large amounts of data on a fairly continuous basis. Moreover, the widespread availability of geolocated information over multiple networks such as social networks, service networks, and publishing media networks, enables the rapid identification, mapping, and prediction of an infectious disease or health condition spread through one or more geographic areas.

In the field of disease diagnostics, one of the relevant problems is to reduce the number of false test results, either positive or negative. False positives may incur undue costs, both monetary and physiological, for treatment procedures on patients that do not need them, and critically skew the population health statistics of that region. False negatives may have nefarious consequences for patients, with the extra cost of belated treatment and associated liabilities for the healthcare provider (personnel and institutions alike). In addition to this, many diagnostic instruments include cartridges configured to provide tests for multiple analytes in the same workflow. Therefore, when a subject walks into a clinic or testing site, unless there are specific reasons to choose a given test, it may be difficult to assess which test is more likely to render useful results. As a result, and in the absence of better discriminators, a clinician may decide to run multiple tests, increasing the likelihood of false positives and incurring added costs for the provider or payor. In another scenario, the symptoms of two different diseases may be highly similar, and the clinician may decide to carry the two assay tests in an abundance of caution, again incurring extra cost.

To resolve the above technical problems arising in the field of networked diagnostic instrumentation and medical diagnostics, a system is provided that collects and analyzes real-time population data, environmental data, disease prevalence data, and epidemiology data to generate hyper-localized pre-test and post-test probabilities. As an added benefit, nonlinear regression and artificial intelligence algorithms are provided that create contextual insights and guidance for the diagnostic instrumentation, using the collected data.

In some embodiments, a system may direct health care providers to prescribe the appropriate diagnostic test, select optimal treatment or care pathways, and/or manage operational and cost management aspects. The end users of these insights may be clinicians, administration, or biomedical engineers. The insights might also be given in some form to the employees of medical device vendors, who may then use them to guide support and training of the users, rather than share the results with them directly.

Some embodiments may include an underlying data platform and associated applications or solutions powered by the unique dataset as described above. The data platform captures geographically specific test results from a network of proprietary diagnostic devices and third-party sources, incorporates additional non-medical data sources, and normalizes and creates up to date derivative insights for use by downstream applications. The downstream applications, installed in client devices communicatively coupled with servers and databases in the data platform, use this data to create patient specific, contextual insights and guidance across clinical and operational use cases.

Contextual insights for diagnostics may be delivered via multiple channels, such as the in-use diagnostic instrument itself, a point of care or laboratory equipment, or on separate digital interfaces via an electronic health record (EHR), location information server (LIS), user portal, mobile application, or through push notifications via email, messaging, or text messages.

Some of the advantages provided by embodiments as disclosed herein include a faster and more timely diagnostic assessment. Clinicians often have a lack of time to examine a patient and make a proper diagnostic. Clinicians may also be challenged to find time to stay abreast on local disease prevalence and trends. Accordingly, embodiments as disclosed herein enable clinicians to perform all these tasks timely. Complementing and providing context to a clinician decision also compensates a lack of skilled personnel in the profession. Clinical labor turnover or lack of desired skill levels in a clinical practice can result in lack of real-time diagnostic capability for a patient, and even catastrophic results, in case of an error.

An additional advantage provided by embodiments as disclosed herein includes a cost reduction in the treatment of infectious diseases. Accordingly, embodiments as disclosed herein eliminate unnecessary diagnostic tests or diagnostic time, thus reducing cost to clinical operations and payors.

Data collected using network architectures as disclosed herein may help in the design of effective population screening strategies for infectious diseases. For example, areas of low disease prevalence that generate relatively high levels of false positives may be identified to reduce screening therein and avoid inappropriate resource spending. Currently, to stay abreast of regional trends, clinicians resort to clinical associations, federal or state public health organizations, newsletters, social media, or from peer-to-peer interactions. In embodiments as disclosed herein, a clinical professional may be upgraded of current events and developments by appropriately injecting the information at the right moment in the patient care pathway. A healthcare provider employee may seamlessly stay current, update protocols, or send email communications to staff as disease trends rise and/or fall. Accordingly, embodiments as disclosed herein enable a quick response to changing and quantification of trends, and the ability to translate information into action at the point of care.

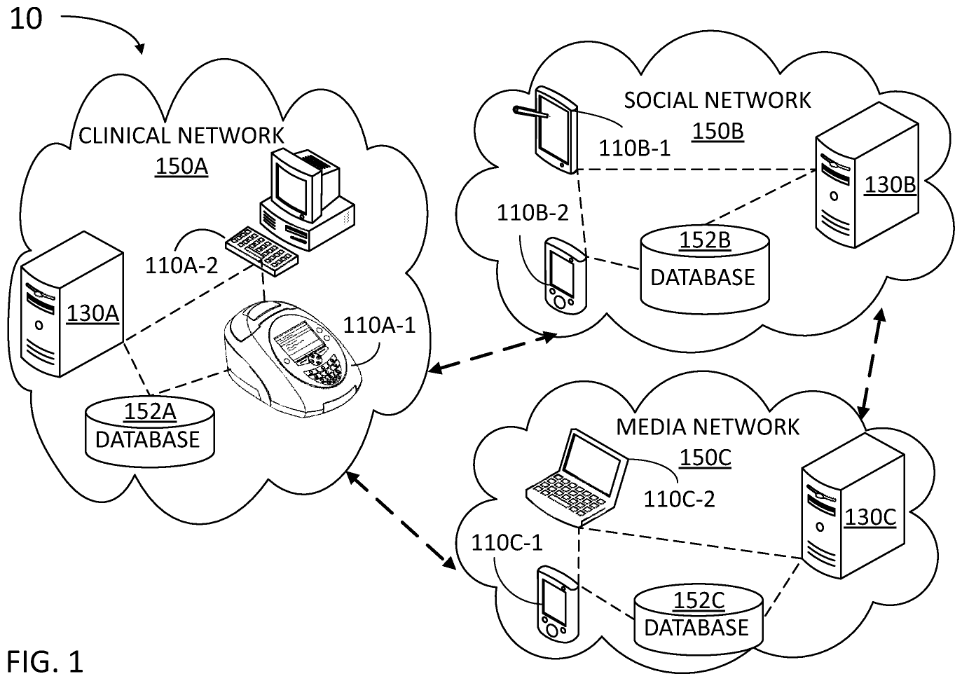

FIG. 1 illustrates an exemplary architecture 10 of a system for visualizing and supporting a contextual diagnostic decision for contagious diseases, according to some embodiments. Architecture 10 includes servers 130A, 130B, and 130C (hereinafter, collectively referred to as “servers 130”), databases 152A, 152B, and 152C (hereinafter, collectively referred to as “databases 152”), and client devices 110A-1 and 110A-2 (“client devices 110A”), 110-B-1 and 110B-2 (“client devices 110B”), and 110C-1 and 110C-2 (“client devices 110C”). Hereinafter, client devices 110A, 110B, and 110C will be collectively referred to as “client devices 110.” Servers 130, databases 152, and client devices 110 are communicatively coupled over a clinical network 150A, a social network 150B, and a media network 150C (hereinafter, collectively referred to as “networks 150”).

Clinical network 150A may include a server 130A, a database 152A, a diagnostic device 110A-1 and other computer and desktop devices 110A-2. Clinical network may group clinical facilities, hospitals, test sites, healthcare providers, and healthcare personnel. In some embodiments, server 130A and database 152A may be hosted by government institutions collecting, updating, and reporting infectious disease data, progress, and outlook. Social network 150B may include any type of social networking service where users of mobile device such as tablets 110B-1, mobile phones 110B-2, and the like, communicate with one another and exchange messages (e.g., health-related comments, symptoms, and the like) hosted by a server 130B and stored in a database 152B. Information handled by server 130B and stored in database 152B is public and may be searched and collected within any one of servers 130 to assess the context of an infectious disease within a given geographical region. Media network 150C may include a server 130C and a database 152C supporting and hosting browsing applications in mobile device 110C-1, laptops 110C-2, and the like. Accordingly, media network 150C may include generic network traffic such as web searches, mobile location information, purchasing information, and the like. In some embodiments, media network 150C may also include weather channel news, and server 130C may thus handle data predicting weather conditions in a geographic area of interest, which is relevant in the context of an infectious disease progression in the geographic area.

One of the many servers 130 and client devices 110 may include a memory storing instructions which, when executed by a processor, cause servers 130 and the client devices 110 to perform at least some of the steps in methods as disclosed herein. In some embodiments, architecture 10 is configured to track diagnostic test results carried over by client devices 110. Upon receiving diagnostic test data from a client device 110, one or more of servers 130 may analyze the data and arrive to a diagnostic, which is stored, together with the raw data collected from client device 110, in databases 152. Client devices 110A in clinical network 150A may include personal, home diagnostic kits that users (e.g., patients or the public in general) may purchase at a pharmacy, a clinic, or order online. For example, in some embodiments, a user may order a test cartridge and a sample collecting disposable and use a personal mobile device to collect the test results from the cartridge (e.g., picture or video), and upload to database 152, for analysis. Additionally, client devices 110A in clinical network 150A may include a diagnostic instrument handled by qualified healthcare personnel at a clinic.

Accordingly, databases 152 may include a data platform that powers downstream applications running in client devices 110 and hosted by servers 130. Data stored in database 152A may include diagnostic data generated from proprietary sources in client devices 110A (e.g., diagnostic instruments having proprietary software applications). The diagnostic data may include test results (positive/negative), patient demographics (age, gender, zip code, and the like), and associated metadata. Data stored in database 152C may include third-party data, such as public health data, web-based symptom checkers, academic databases, social media, payor, provider, or device manufacturer sources. Other data stored in and retrieved from database 152C in media network 150C may include data: disease trends, electronic patient reported symptoms, outcome measurements, digital metrics (e.g., web traffic or search volumes for selected keywords and phrases), and device information or outputs (e.g., from network-coupled thermometer readings, allergen and pollen counts, weather forecasts, and the like). Patient information in architecture 10 and databases 152 is safe, and not personally identifiable information (PII), in that no direct personal information from a consumer is involved (e.g., address, phone number, social security number, and the like).

Servers 130 may include any device having an appropriate processor, memory, and communications capability for hosting the history log, a diagnostic database, and a healthcare provider host. The healthcare provider host may be accessible by multiple client devices 110 over networks 150. In some embodiments, servers 130 may include a social network host, or a network service provider such as a search engine. Client devices 110 may include, for example, diagnostic instruments, desktop computers, mobile computers, tablet computers (e.g., including e-book readers), mobile devices (e.g., a smartphone or PDA), or any other devices having appropriate processor, memory, and communications capabilities for accessing one or more of servers 130 through network 150. In some embodiments, client devices 110 may include a Bluetooth radio or a near-field-communication (NFC) transmitter device and application, enabling the client device to communicate directly with another device in its proximity, e.g., a device at a point-of-sale (POS) in a retail store. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

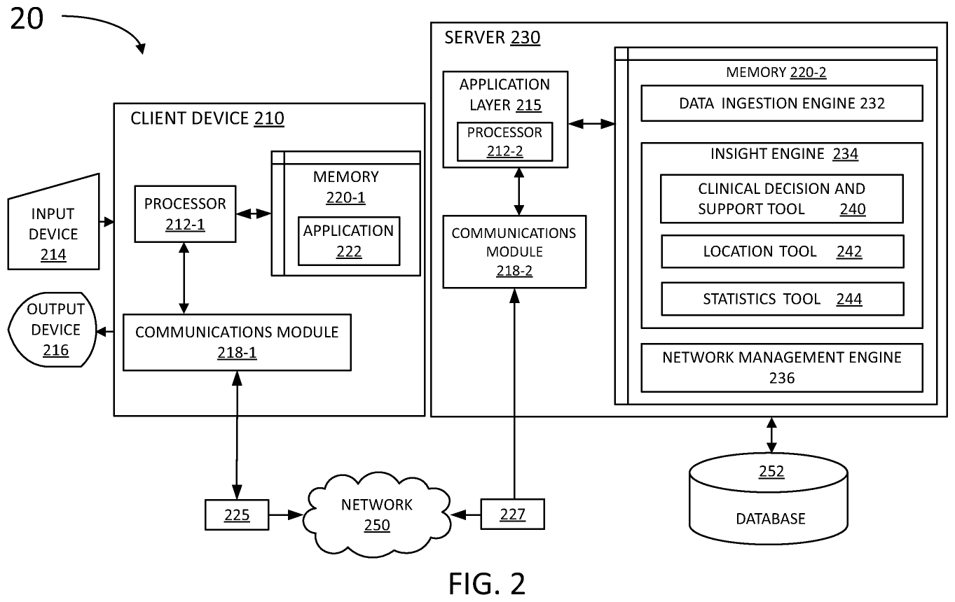

FIG. 2 is a block diagram 20 illustrating a client device 210 and a server 230 in an architecture for providing contextual geolocated information in a network architecture of diagnostic instruments for infectious disease detection (e.g., architecture 10), according to some embodiments. Client device 210 may include any one of: a diagnostic instrument, a mobile device, a computer (e.g., desktop, laptop, palm device), a wearable device attached to the body of the user, a virtual reality/augmented reality headset or wearable device, or any combination thereof. For example, in some embodiments, a diagnostic instrument may couple to network 250 autonomously, or may be paired to a mobile device with the user. A user of client device 210 may include physicians, nurses, lab managers, lab technicians, patients, vendors, and people from the public handling a diagnostic instrument (e.g., at home). Client device 210 and server 230 are communicatively coupled with each other with via network 250, through communications modules 218-1 and 218-2 (hereinafter, collectively referred to as “communications modules 218”). Communications modules 218 are configured to interface with network 250 to send and receive information, such as data, requests, responses, and commands to other devices on network 250. In some embodiments, communications modules 218 can be, for example, modems or Ethernet cards. Client device 210 may be coupled with an input device 214 and with an output device 216. Input device 214 may include a keyboard, a mouse, a pointer, or even a touchscreen display that a user (e.g., a consumer) may utilize to interact with the client device. Likewise, output device 216 may include a display and a speaker with which the user may retrieve results from client device 210. In some embodiments, input device 214 includes a sample carrying cartridge for a diagnostic test. The sample carrying cartridge may include one or more assays for testing multiple analytes along separate media tracks. Input device 214 may also include a light source configured to illuminate the sample carrying cartridge and excite an emission (e.g., fluorescence) or absorption from one or more target locations in the media tracks. Output device 216 may include a camera configured to capture an image or video of the sample carrying cartridge and the emission or absorption at the target locations. Client device 210 may provide a data packet 225 to server 230. In embodiments where client device 210 is a mobile device or desktop, input device 214 and output device 216 may include diagnostic devices themselves. In some embodiments, input device 214 and output device 216 could also be receiving the laboratory order digitally and directly from the device via way of an EHR, LIS, or other clinical information system, or a direct connection to the clinical user interface. In embodiments where client device 210 is a diagnostic device, input device 214 and output device 216 may be a display for a GUI screen to input the sample ingestion module.

Data packet 225 may include the image or video of the light emission or absorption at the target locations in the sample carrying cartridge, according to some embodiments. Data packet 225 may also include a geolocation information associated with the position of client device 210. In some embodiments, data packet 225 includes a query to retrieve infection data rates at the location of client device 210 from database 252. Data packet 225 may also include probability scores, derivative insights, clinical decision guidelines/rules, error messages, and troubleshooting-all before, during, or after the test is run. Data packet 225 could include test results (e.g., positive or negative or a quantified analyte level). Data packet 225 could also be consistent with the existing language, where the diagnostic device is simply taking an image or raw diagnostic of the sample, and then sending data packet 225 to the cloud to generate the test result in a cloud-based application. When client device 210 is one of multiple diagnostic devices in a test network, data packet 225 joins a swarm of similar data packets 225 transmitted to server 230 each diagnostic test device for processing in insight engine 234.

Each of client device 210 and server 230 may include processors 212-1 and 212-2, and memories 220-1 and 220-2, respectively (hereinafter, collectively referred to as “processors 212” and “memories 220”). Memories 220 may store instructions which, when executed by processors 212, cause servers 230 and devices 210 to perform, at least partially, some of the procedures in methods as disclosed herein.

Processors 212 may be configured to perform normalization and standardization of data packet 225 regardless of the type or proprietary details of client device 210 and current formatting of application 222. In some embodiments, data packet 225 may include geolocation information linking diagnostic results to specific areas, potentially zip code or geofenced latitude/longitude.

In some embodiments, server 230 may provide to client device 210 a data packet 227. Data packet 227 may include software and updates for an application 222 running in client device 210 and hosted by server 230. In some embodiments, data packet 227 may include an epidemiologic report for the local area for client device 210, provided upon request by application 222, or on a scheduled basis, or as an infectious disease alert sent from server 230 to one or more client devices 210 in network 250. Data packet 227 may also include probability scores, derivative insights, clinical decision guidelines/rules, insights and recommendations, error messages, and troubleshooting. In some embodiments, a clinical insight/recommendation may be generated by insight engine 234, and client device 210 only acts as an agent to take the user test request, and then reaches out to server 230 to review the proposed test sample against the current at-risk disease types. Data packets 225 and 227 may include real-time data and predictive data. Predictive data may include predictions based on algorithms on future trends and use that to influence the output. In some embodiments, data packets 225 and 227 may include a registry of known diagnostic tests and their associated performance, including sensitivity and specificity.

In some embodiments, server 230 may install and host application 222 in memory 220-1 of client device 210, via an application layer interface (API) 215. API 215 may provide operational management capabilities to users of client device 210 (e.g., physicians, pharmacists, lab managers, administration, quality control personnel) processed by an insight engine 234 and a network management engine 236 in memory 220-2. API 215 has access to specific calls and services for memory 220-2. In some embodiments, client device 210 can initiate a call to API 215 at specific triggers for updates on disease prevalence, decision guidelines, or customer support, and the like.

Application 222 may be a diagnostic assay application configured to run a diagnostic test in client device 210. Accordingly, application 222 may be configured to display instructions to the user (e.g., a healthcare professional in a clinic or a patient at home) in a display (e.g., output device) 216. In that regard, application 222 may display for the user corrective actions needed for the diagnostic test to proceed. Application 222 may also request input from the user, such as metadata (name, date, location, symptoms, and desired test), prior to, during, or after the performance of a diagnostic test.

Application 222 may include a graphic user interface (GUI) coupled to output device 216. Application 222 may be a location application that locates a point of care or laboratory device (e.g., geocoordinates, zip code, address, building, floor and room number, and the like). Application 222 may include communication to the user regarding pre-test procedures such as displaying contextual information on pre-test probability and recommendations on clinical or operational appropriateness for a requested test. Application 222 may also provide post-test communications to the user. Some post-test communications may include post-test probabilities displayed alongside the test result and operational or clinical guidance, such as the need for reflex testing using molecular or a higher performance diagnostic. In some embodiments, application 222 may periodically query server 230 and/or database 252 for updates on the local progress of an infectious disease, or an epidemiologic report in the area. This ensures the user that the test to be run in client device 210 will be used for a diagnostic of an infectious disease that is prevalent in the area.

A data ingestion engine 232 controls and manages the data collection (e.g., of data packets 225) from multiple client devices 210 via network 250. Insight engine 234 may include a clinical decision and support tool 240, a location tool 242, and a statistics tool 244.

Clinical decision and support tool 240 includes an engine focused on guiding the right test selection, interpretation of the test, recommending additional tests, and recommending courses of therapy and requesting additional information in-process to help support the recommendation. In some embodiments, clinical decision and support tool 240 includes an engine focused on operations and logistics. For example, clinical decision and support tool 240 may determine which tests may be in demand, evaluate inventory levels at an account, evaluating inventory levels at one hospital versus another hospital in the same network and suggesting balancing, identifying users who constantly deviate from best practices and alerting administration of such cases.

Clinical decision and support tool 240 evaluates a requested test and may prevent client device 210 or alert the user to prescribe specific diagnostic tests based on prevalence of an infectious disease and a location information provided by location tool 242. For example, when flu is not prevalent in the location where a test is requested, clinical decision and support tool 240 may prevent client device 210 from running a flu test. Rather, clinical decision and support tool 240 may recommend a different (e.g., more appropriate, or likely to have more effective results) type of test (e.g., molecular or antigen), based on known test performance against prevalence, risk of disease, and rate of transmission. Administrative users of the client device 210 (e.g., a diagnostic device) may have the option on how much to limit workflow based on disease prevalence. For example, client device 210 may prevent a lab technician from running a test where there is no prevalence. In some embodiments, clinical decision and support tool 240 provides specific guidance on the appropriate test for a subject based subject healthcare history via clinical notes and natural language processing, and/or symptoms selected via a form, in combination with the contextual disease data to provide a suggested diagnostic test. In some embodiments, clinical decision and support tool 240 may cause client device 210 to display a message such as “Based on the subject healthcare history provided, and regional disease prevalence and other data, we expect this subject to be 76% likely to test positive for RSV. Please press here to proceed with testing to confirm.”

In some embodiments, client device 210 may issue alerts that pop up in the display 216 and can be overridden. In some embodiments, users of client device 210 may indicate a disease prevalence against the test in the device log for future analysis (e.g., to be transferred to database 252). In some embodiments, when a local prevalence is less than a pre-selected threshold for an infectious disease that may be more prevalent elsewhere, clinical decision and support tool 240 may have a “safety net” so that a local level of testing is maintained (e.g., every 5^{th} test request from client device 210, the request is approved). More generally, clinical decision and support tool 240 supports users of client device 210 (e.g., physicians, nurses, pharmacists, and the public) in clinical decision making. In some embodiments, clinical decision and support tool 240 guides appropriate clinical workflow for the patient (e.g., the subject of a diagnostic test performed with client device 210). For example, clinical decision and support tool 240 may evaluate patient history (e.g., retrieved from database 252), symptoms, and other input. Clinical decision and support tool 240 may also score pre-test probability and suggest appropriate diagnostic tests, sampling sites, and other desirable inputs. In addition, clinical decision and support tool 240 may provide after-test results, compare against post-test probability (in combination with statistics tool 244), and recommend additional diagnostic tests. Clinical decision and support tool 240 interprets diagnostic results, patient information data, and location information (in combination with location tool 242) and recommends an appropriate care pathway. In some embodiments, clinical decision and support tool 240 relays test results to database 252. Clinical decision and support tool 240 provides support to the users of client device 210 (e.g., layperson consumers, either for user or dependents, and the like), based on location information retrieved by location tool 242. In some embodiments, clinical decision and support tool 240 pushes notification purchase recommendations for tests when disease trends rise or are predicted to rise above a certain threshold, including discounts or purchase incentives, by transmitting a message (e.g., e-mail, chat, and the like) to client device 210. Clinical decision and support tool 240 may communicate with client device 210 in the context of a diagnostic text, or more generally, in the context of an outbreak or a monitoring for an infectious disease (whether the user of client device 210 is planning to take a test or not). Accordingly, clinical decision and support tool 240 may transmit messages to client device 210 for symptom checking and interpretation against local prevalence of an infectious disease and suggesting potential tests or care pathways based on pre-test probability. In some embodiments, clinical decision and support tool 240 may refer the user to a virtual care flow based on test results and a higher probability of positive diagnostics. In some embodiments, clinical decision and support tool 240 includes a virtual assistant that interacts with the user in real time. For example, in some embodiments, clinical decision and support tool 240 may take the user of client device 210 into a virtual reality room for a one-on-one support session.

Location tool 242 handles location information via a companion mobile application (e.g., application 222) in an administrator portal, embedded in an EHR server (e.g., server 230) or diagnostic device (e.g., client device 210). Clinical decision and support tool 240 may collaborate with location tool 242 to find out that a user of client device 210 is travelling into or out of a region where a certain infectious disease is prevalent. Thus, clinical decision and support tool 240 may send a message to the user that a diagnostic test would be advisable. Additionally, clinical decision and support tool 240 may schedule calendars and provide reminders to the users of client device 210 for taking a diagnostic test. In some embodiments, clinical decision and support tool 240 may collaborate with statistics tool 244 to find a pre-test probability rate. When the pre-test probability rate is above a pre-selected threshold, clinical decision and support tool 240 generates a prescription for a diagnostic test, either synchronously or asynchronously.

Statistics tool 244 performs statistical operations based on historical data (e.g., EHR in database 252), and other data collected from network resources (e.g., location data, infectious disease progress, and the like). Statistics tool 244 performs mathematical analysis such as averages, variance, standard deviation and higher order moments of a distribution, histograms, fit to probability functions, and the like. In addition, and in collaboration with clinical decision and support tool 240, statistics tool 244 may interpret test results using post-test probability to offset false positives, predict future disease trends in a geography, and guide event planning and screening requests.

In some embodiments, network management engine 236 controls data inputs 225 related to web traffic through network 250 that may not be directly associated with disease, diagnostic, or even technical healthcare data, but may create the insights and guidance for disease diagnostic and care. Some of the inputs handled by network management engine 236 may include population data (e.g., age, gender, socioeconomic information), social, environmental information, third-party clinical information, social networks, and the like. In such cases, client device 210 may include a mobile phone or any other network computer with which the user communicates with network 250. For example, in some embodiments, the user of client device 210 performs a search query for medication or pharmacies, or items to alleviate cough symptoms, or asks in a social network about certain symptoms or conditions. All this information may be collected in a data packet 225 and captured or selected by network management engine 236.

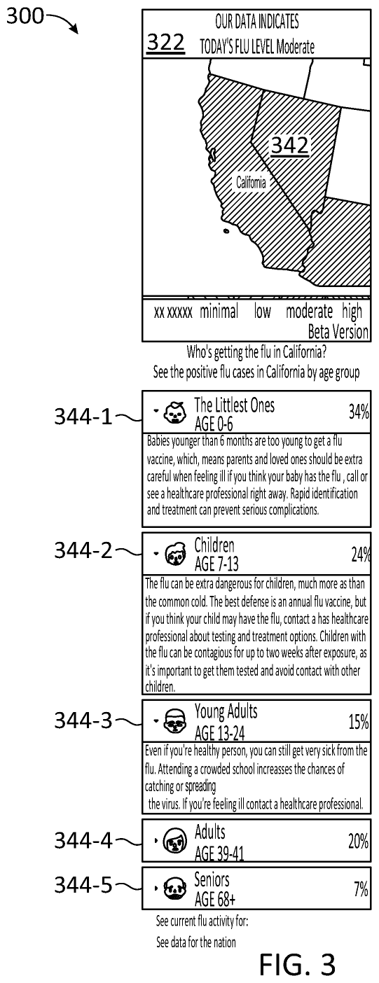

FIG. 3 illustrates a feedback 300 based on geolocated information 342 associated with a contagious disease in a network, according to some embodiments. An application 322 displays on a mobile device from a user geolocated information 342 aided by statistical data 344-1, 344-2, 344-3, 344-4, and 344-5 (hereinafter, collectively referred to “statistical data 344”). Statistical data 344 is a breakup of tests carried out for different demographic sectors (e.g., by age) and relative percentages.

Application 322 highlights who is getting the disease but not necessarily the probability of testing positive or negative. Application 322 could be a consumer facing application for population health guidance, which users can access via mobile devices, desktops, or any other networked computer. Application 322 may also provide the users with recommendations as to which at-home tests should be acquired or applied and provide links to virtual or brick-and-mortar test providers.

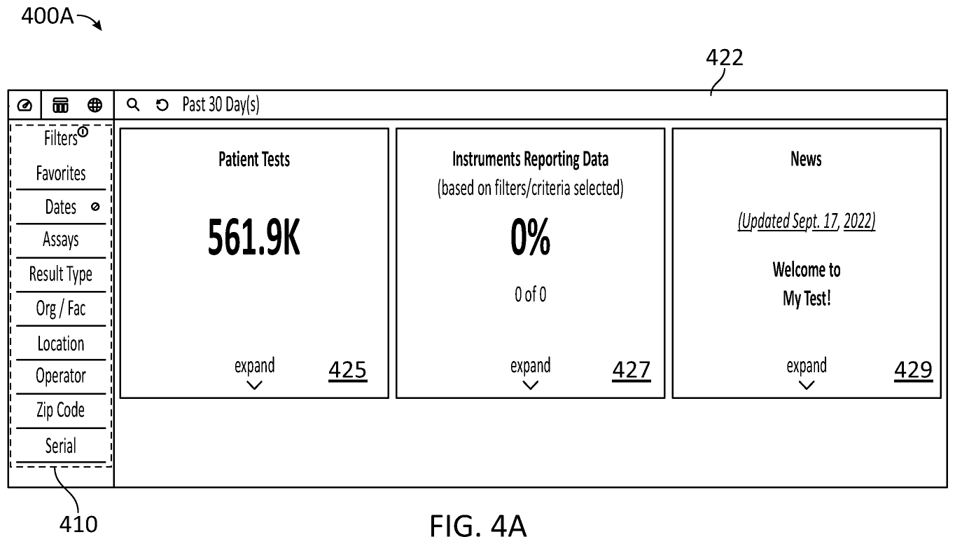

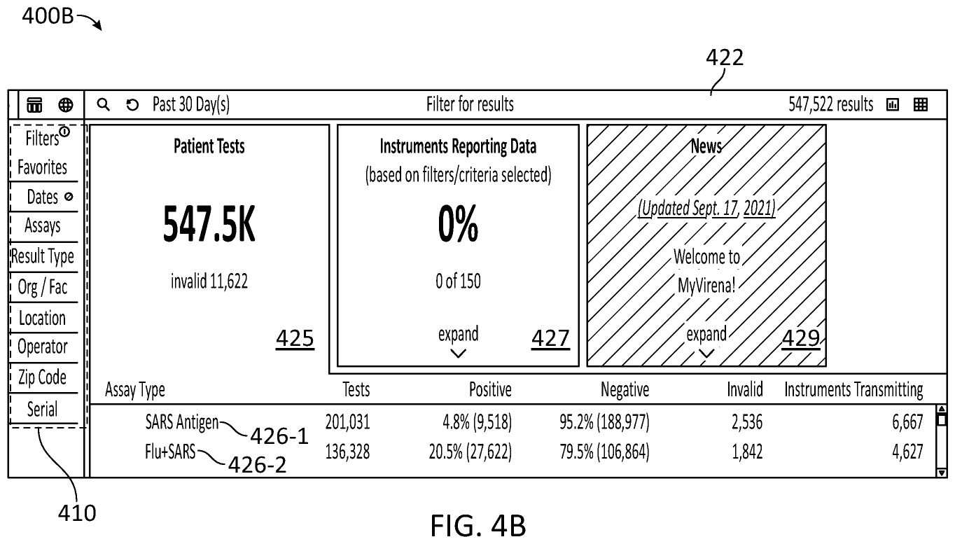

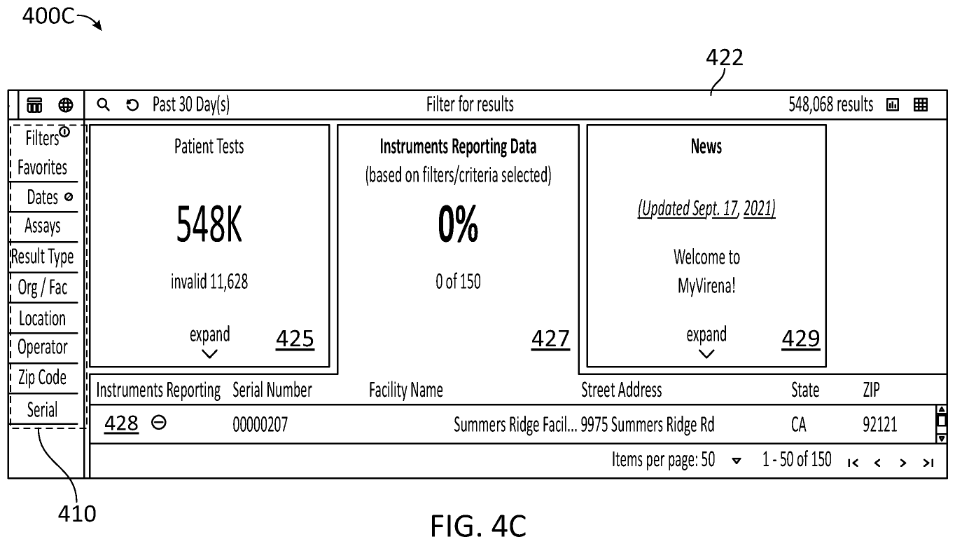

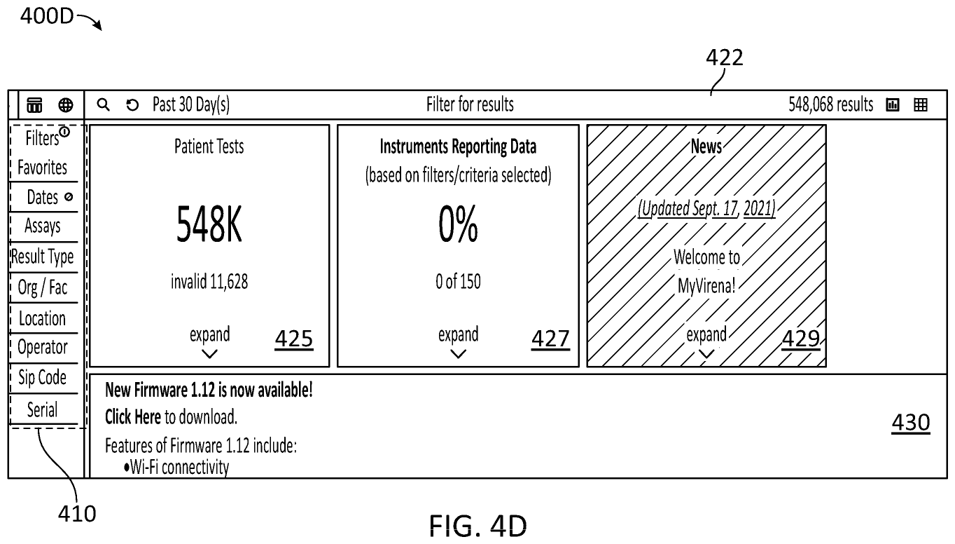

FIGS. 4A-4D illustrate different screenshots 400A, 400B, 400C, and 400D (hereinafter, collectively referred to as “screenshots 400”) from a webpage 422 hosted by a server in a system for visualizing and supporting a contextual diagnostic decision for contagious diseases, according to some embodiments. Screenshots 400 are a visual representation of different products of an insight engine, which would then be pushed to user applications running in client devices (e.g., insight engine 234 hosting applications 222 and 322, and client device 210). The insight engine generates screenshots 400 by automating visualization and analytics before turning them into more useful context recommendations at the point of care. Screenshots 400 could represent an administrative tool viewable by biomedical engineers and lab managers, other than clinicians.

Webpage 422 includes a menu 410 where users can select from different tools such as filters, dates, assays (e.g., test assays available for selected infectious diseases), result types, organization/facility, location, operator, zip code, and serial number (e.g., serial number of a diagnostic instrument and the like). Different tabs may include patient tests 425, instrument reporting data 427, and notifications 429.

Screenshot 400B illustrates patient tests 425 including a list of test assays 426-1 (SARS Antigen) and 426-2 (FLU+SARS), hereinafter, collectively referred to as “test assays 426.”

Screenshot 400C illustrates instrument reporting data 427 including a list of facilities 428.

Screenshot 400D illustrates notifications 429 including firmware updates 430, and other items that users may download into a diagnostic instrument or a service station.

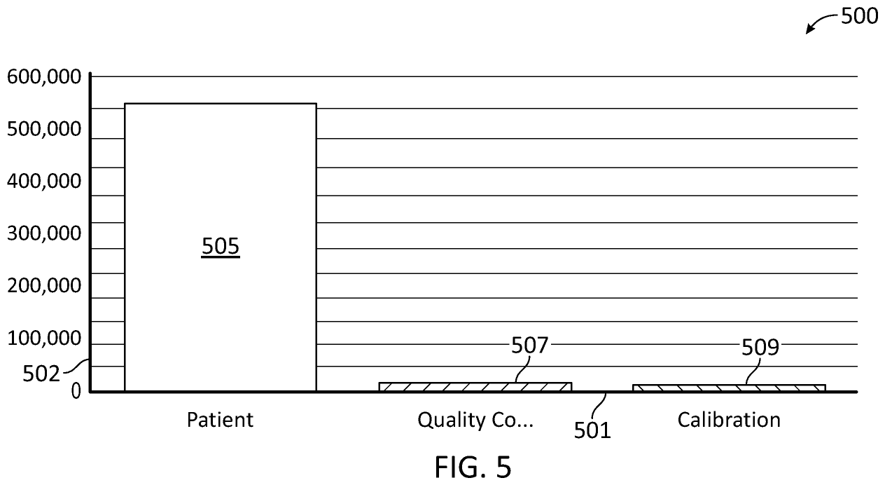

FIG. 5 illustrates a bar chart 500 indicative of numbers 502 of test types 501 performed in a network of diagnostic instruments, according to some embodiments. Test types 501 may include patient diagnostics 505, quality control 507, and calibration tests 509.

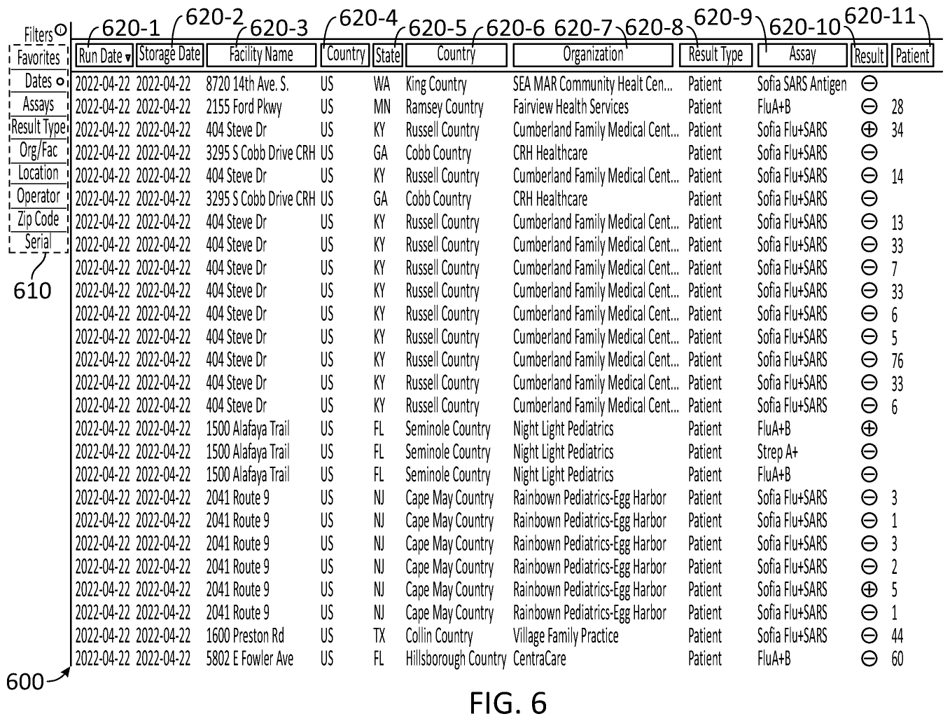

FIG. 6 illustrates a table 600 including data for multiple diagnostic tests, patients, and locations in a network of diagnostic instruments (cƒ. networks 150 and 250), according to some embodiments. Table 600 may be selected from menu 610 in a website (e.g., menu 410 in website 422). Each line in table 600 is associated with a diagnostic test performed at a given location and time for a specified array, and with a specified result.

Table 600 includes columns 620-1 (run date), 620-2 (storage date), 620-3 (facility name), 620-4 (country), 620-5 (state), 620-6 (county), 620-7 (organization), 620-8 (result type), 620-9 (assay), 620-10 (result), and 620-11 (patient), hereinafter, collectively referred to as “columns 620.”

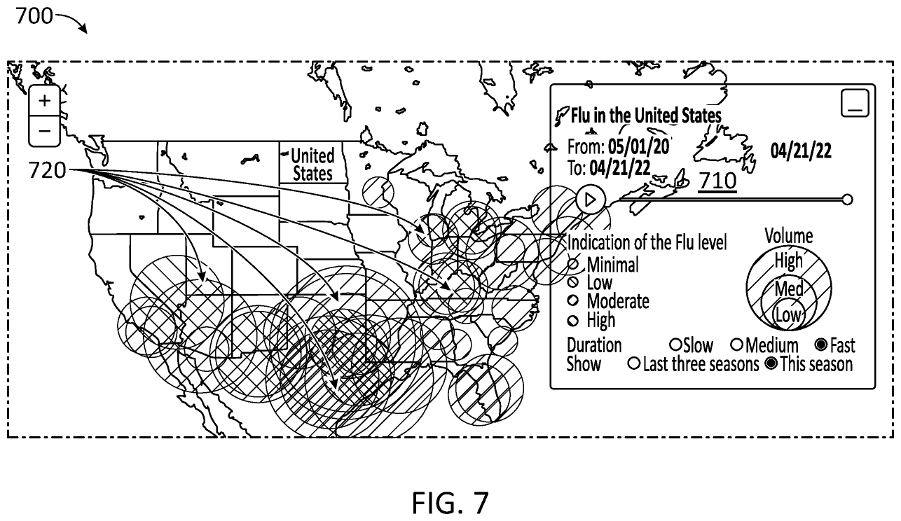

FIG. 7 illustrates a map 700 including a sequence 710 indicative of the spread of an infectious disease over different portions within a large geographical area, for a selected span of time, according to some embodiments. Sequence 710 is an animated sequence of frames illustrating, when played, the geographic progression of an infectious disease over multiple hotspots. Each of circles 720 in map 700 is centered in a city, town, or locality, and its diameter indicates a total number of positive diagnostics of the disease, in the center. In some embodiments, multiple circles may overlap when the disease grows in contiguous localities, providing further graphic illustration of the infectious density of the disease.

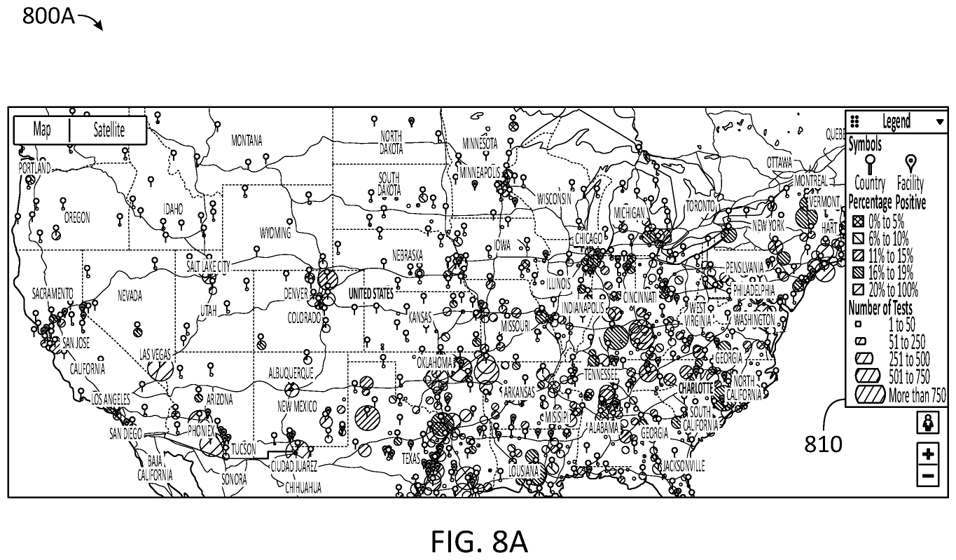

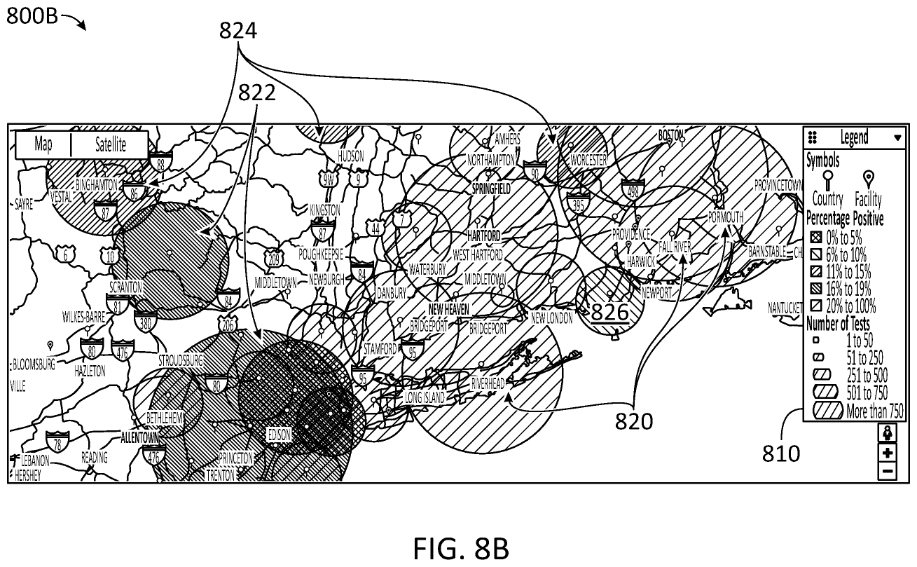

FIGS. 8A-8B are maps 800A and 800B (hereinafter, collectively referred to as “maps 800”) retrieved from a webpage (e.g., webpage 422) hosted by a server in a system for visualizing and supporting a contextual diagnostic decision for contagious diseases, according to some embodiments.

Maps 800 include a key 810 indicating different features: a color code illustrates a percentage of positive cases in each area: Circles 820 (20-100%), 822 (16-19%), 824 (11-15%), and 826 (6-20%) are centered on a county seat. Maps 800 include indicators for county and facility.

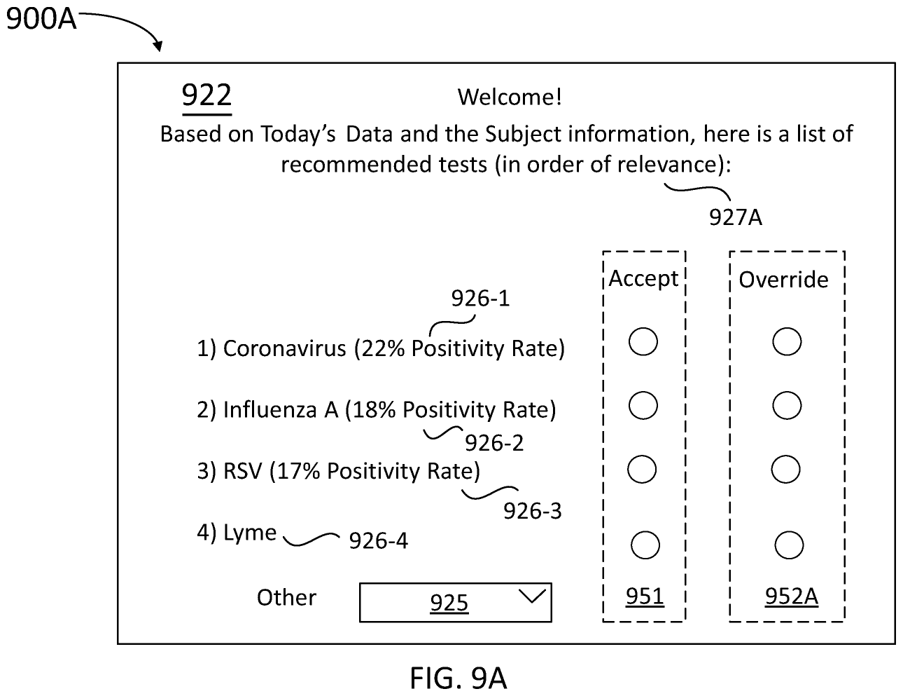

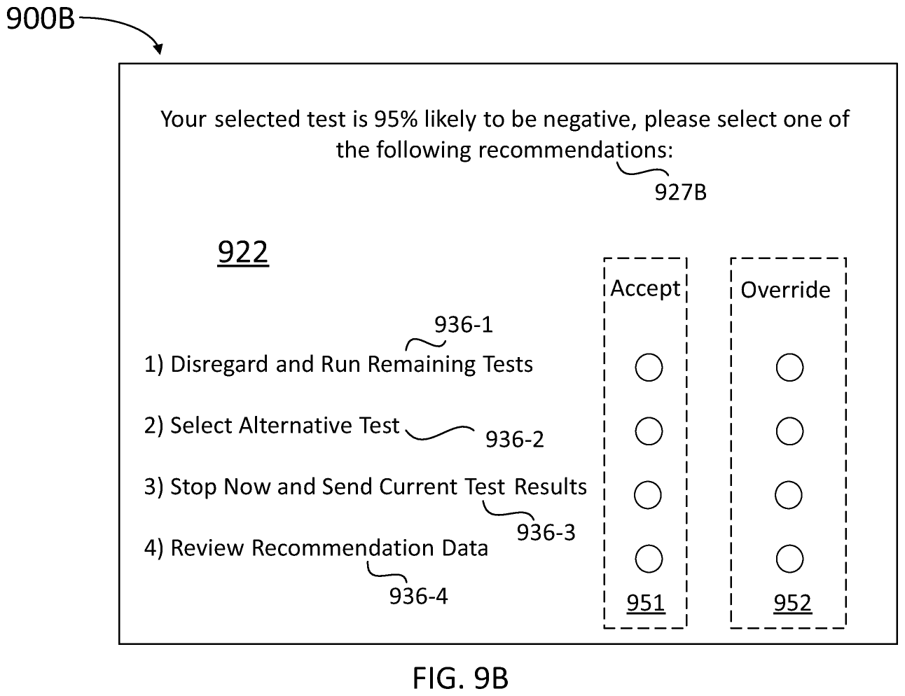

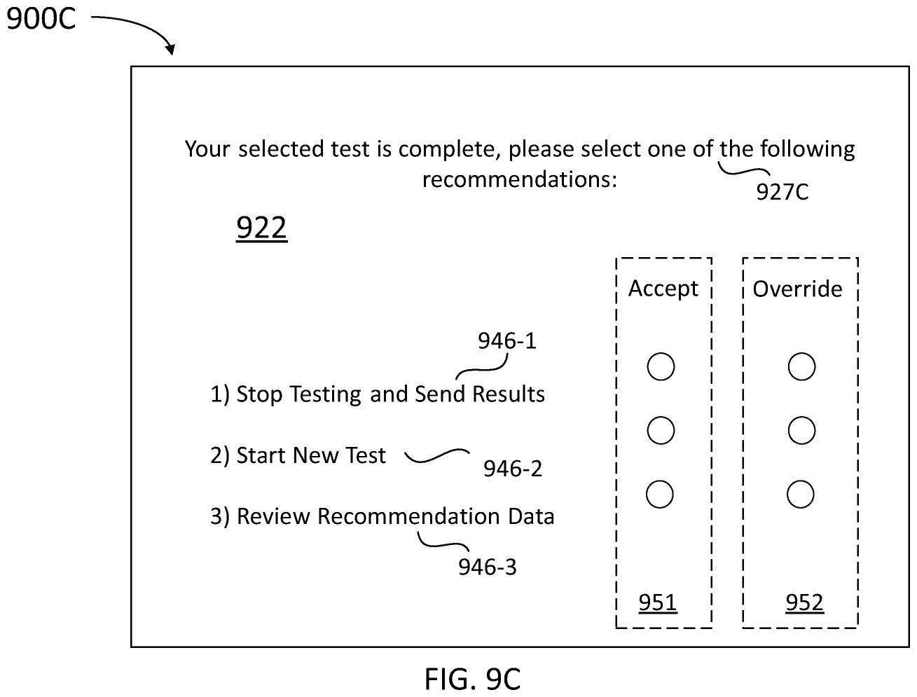

FIGS. 9A-9C are screenshots 900A, 900B, and 900C (hereinafter, collectively referred to as “screenshots 900”) of an application 922 installed in a diagnostic device including messages 927A, 927B, and 927C (hereinafter, collectively referred to as “messages 927”) to the user, according to some embodiments. Each of messages 927 includes a set of options that the user can accept 951, or override 952A, 952, any of the options. Screenshots 900, or variants consistent with the present disclosure may also be present at a prescribing step (as opposed to the operational test or running step), where the appropriate prescribing clinician (typically a physician, but also a PA, pharmacist, CNP, and the like) would be guided on appropriate tests. Screenshots 900 may appear as a popup in an HER application (e.g., application 922), or printed out on patient charts as part of the hospital rounding packaging, at the nursing station input screen, or as part of the lab manager order check/QC check.

Message 927A may include an initial message (e.g., “Welcome! Based on Today's Data and the Subject information, here is a list of recommended tests (in order of relevance)”). The recommended tests (hereinafter, collectively referred to as “recommended tests 926”) may be: 1) Coronavirus (22% positivity rate, 926-1); 2) Influenza A (18% positivity rate, 926-2), 3) Respiratory Syncytial Virus (RSV, 17% positivity rate, 926-3); and 4) Lyme (926-4). The user may override 952A all suggestions, and enter another test 925 to perform, in case of an override 952A. Recommended tests 926 are selected based on the geographic location of the diagnostic device, and the evolution and likelihood of a positive result for any one of the recommended infectious diseases. In addition, certain initial data that the user may have input into the diagnostic device upon logging-in may be relevant, such as demographic data (e.g., age, gender, occupation, and the like). Other relevant subject information may include initial tests such as temperature, symptom descriptions, or even a perfunctory visual analysis of the subject (e.g., red eyes, skin rashes, and the like).

Message 927B may include a text provided before completion of the diagnostic assay, at a point where a threshold confidence level indicates a negative result (or positive result, as the case may be, without limitation: “Your selected test is 95% likely to be negative, please select one of the following recommendations”). The recommendations (hereinafter, collectively referred to as “recommendations 936”) may include: 1) Disregard and run remaining tests (936-1); 2) Select alternative test (936-2); 3) Stop now and send current test results (926-3); and 4) Review recommendation data (936-4).

Message 927C may include a text provided after the assay is completed, or any of the options from message 927B were accepted: “Your selected test is complete, please select one of the following recommendations” (hereinafter, collectively referred to as “recommendations 946”). The recommendations may include any one or more, of: 1) Stop testing and send results (946-1); 2) Start new test (946-2); and 3) Review recommendation data (946-3).

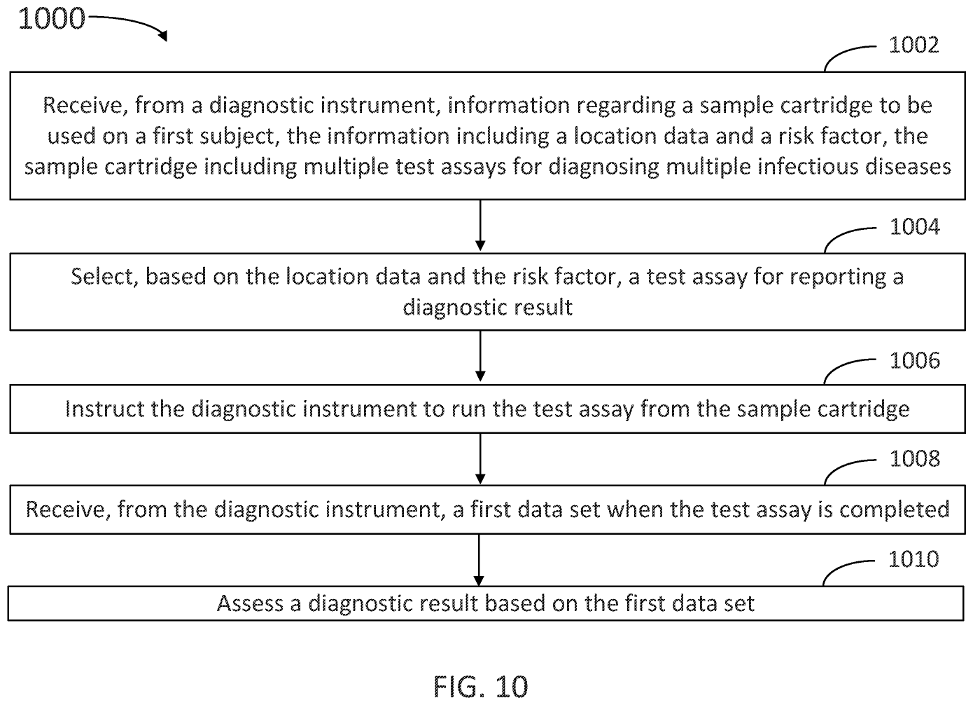

FIG. 10 is a flow chart illustrating steps in a method 1000 for visualizing and supporting a contextual diagnostic decision for contagious diseases, according to some embodiments. Method 1000 may be performed at least partially by any one of the plurality of servers in collaboration with one or more client devices and databases, communicatively coupled through a network, as disclosed herein (cƒ. client devices 110 and 210, servers 130 and 230, databases 152 and 252, and networks 150 and 250). For example, at least some of the steps in method 1000 may be performed by one component in an architecture (cƒ. architectures 10 and 20), including a mobile device running code for a browser and an application to access a website for an insight engine that processes logic to evaluate and contextualize an infectious disease outbreak and a network management engine (e.g., insight engine 234 and network management engine 236). In some embodiments, the insight engine may include a clinical decision and support tool, a location tool, and a statistics tool, as disclosed herein (cƒ. clinical decision and support tool 240, location tool 242, and statistics tool 244). In some embodiments, one or more of the servers may also include an application layer to host and handle an application installed in a client device (e.g., application layer 215), so third-party users may access the disease analysis engine. Accordingly, at least some of the steps in method 1000 may be performed by a processor executing commands stored in a memory of one of the servers or client devices, or accessible by at least one of the servers or client devices (e.g., processors 212 and memories 220). Further, in some embodiments, at least some of the steps in method 1000 may be performed overlapping in time, almost simultaneously, or in a different order from the order illustrated in method 1000. Moreover, a method consistent with some embodiments disclosed herein may include at least one, but not all, of the steps in method 1000.

Step 1002 includes receiving, from a diagnostic instrument, information regarding a sample cartridge to be used on a first subject, the information including a location data and a risk factor for at least one of multiple infectious diseases, the sample cartridge including multiple test assays for diagnosing the infectious diseases. In some embodiments, step 1002 includes receiving, from a search engine, a datum associated with a search frequency for a selected keyword associated with an infectious disease in an area associated with the location data. In some embodiments, step 1002 includes providing, to the diagnostic instrument, an update for an application interface running the diagnostic instrument, based on the information regarding the sample cartridge and an identifier of the diagnostic instrument. In some embodiments, step 1002 includes receiving patient history via clinical notes and natural language processing, including symptoms selected via a form filled by the subject or a clinician.

Step 1004 includes selecting, based on the location data and the risk factor, a test assay for reporting a diagnostic result. In some embodiments, step 1004 includes providing to the subject or clinician a specific guidance on the appropriate test to pursue. In some embodiments, step 1004 includes determining a false positive probability above a pre-selected threshold on the diagnostic result for the test assay. In some embodiments, step 1004 includes preventing the diagnostic instrument from running a test assay based on the location data and the subject symptom. In some embodiments, step 1004 includes determining an infectious disease prevalence associated with the location data and the test assay. In some embodiments, step 1004 includes communicating to a client device associated with the location data, a request to run a test assay from a sample cartridge in the diagnostic instrument.

Step 1006 includes instructing the diagnostic instrument to run the test assay from the sample cartridge. It will be appreciated that the test assay, or plurality of test assays, on the sample cartridge can be any format of a test assay capable of determining presence or absence of an analyte of interest in a sample from a patient or subject. Exemplary, non-limiting test assays include immunoassays, including lateral flow immunoassays and enzyme-linked immunosorbent assays, and molecular assays for detection of genetic material, such as detection of nucleic acid (DNA and/or RNA) using a single molecule detection method, such as a biosensor, or using an amplification technique such as polymerase chain reaction (PCR) amplification or isothermal amplification.

Step 1008 includes receiving, from the diagnostic instrument, a first data set when the test assay is completed.

Step 1010 includes assessing a diagnostic result based on the first data set. In some embodiments, step 1010 includes retrieving, from a database, a second data set associated with a completed test assay for a second subject with a validated diagnostic result and comparing the first data set with the second data set. In some embodiments, step 1010 includes providing a virtual assistant for the user based on the diagnostic result. When the first test result is negative, and there are multiple high risk diseases, step 1010 includes providing the user with a recommendation to test the next highest probability analyte.

In some embodiments, step 1010 may include displaying a recommendation to a user of the diagnostic instrument based on the diagnostic result. The recommendation may include an optimal diagnostic approach (e.g., alternative or complementary diagnostics that may be available to the subject, based on initial diagnostic results). In some embodiments, step 1010 may include recommending a reflex test for the subject, monitoring the subject (e.g., further tests on a regular schedule), a physical examination, and a review of subject healthcare history. In some embodiments, step 1010 may be triggered based on low disease probabilities against a positive result, or when a user runs a diagnostic for the same low probability analyte twice on a subject and gets a positive result each time. In some embodiments, step 1010 includes displaying a care pathway for the first subject when the diagnostic result is positive for an infectious disease.

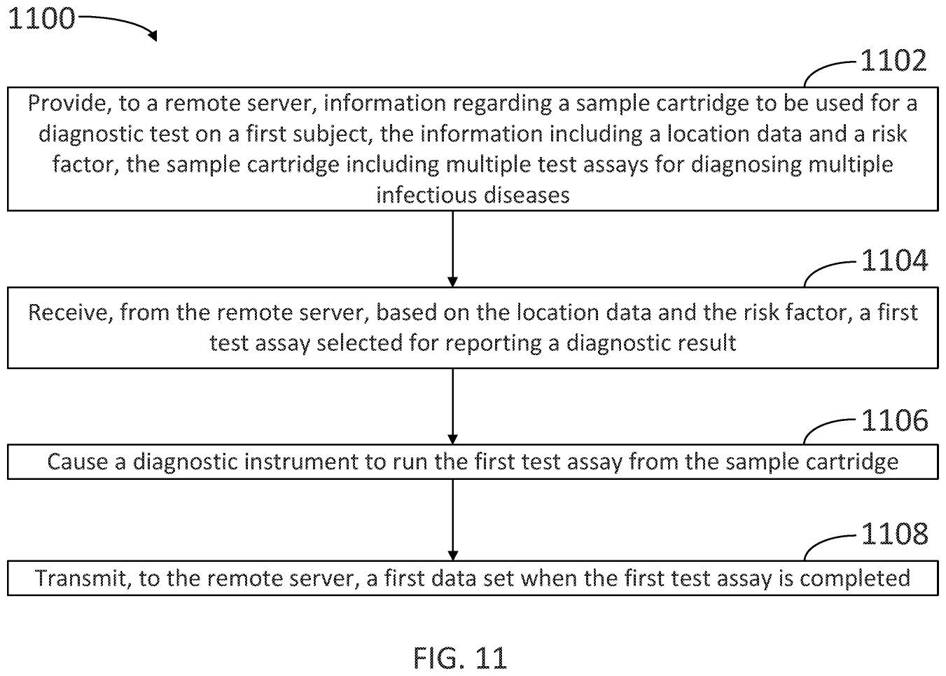

FIG. 11 is a flow chart illustrating steps in a method 1100 for performing a diagnostic test for an infectious disease, according to some embodiments. Method 1100 may be performed at least partially by any one of the plurality of servers in collaboration with one or more client devices and databases, communicatively coupled through a network, as disclosed herein (cƒ. client devices 110 and 210, servers 130 and 230, databases 152 and 252, and networks 150 and 250). For example, at least some of the steps in method 1100 may be performed by one component in an architecture (cƒ. architectures 10 and 20), including a mobile device running code for a browser and an application to access a website for an insight engine that processes logic to evaluate and contextualize an infectious disease outbreak and a network management engine (e.g., insight engine 234 and network management engine 236). In some embodiments, the insight engine may include a clinical decision tool, a location tool, a statistics tool, and a consumer support tool, as disclosed herein (cƒ. clinical decision and support tool 240, location tool 242, and statistics tool 244). In some embodiments, one or more of the servers may also include an application layer to host and handle an application installed in a client device (e.g., application layer 215), so third-party users may access the disease analysis engine. Accordingly, at least some of the steps in method 1100 may be performed by a processor executing commands stored in a memory of one of the servers or client devices, or accessible by at least one of the servers or client devices (e.g., processors 212 and memories 220). Further, in some embodiments, at least some of the steps in method 1100 may be performed overlapping in time, almost simultaneously, or in a different order from the order illustrated in method 1100. Moreover, a method consistent with some embodiments disclosed herein may include at least one, but not all, of the steps in method 1100.

Step 1102 includes providing, to a remote server, information regarding a sample cartridge to be used for a diagnostic test on a first subject, the information including a location data and a risk factor for at least one of multiple infectious diseases, the sample cartridge including multiple test assays for diagnosing the infectious diseases. In some embodiments, step 1102 includes requesting, from the remote server, an epidemiology report for an infectious disease associated with the location data.

Step 1104 includes receiving, from the remote server, based on the location data and the risk factor, a first test assay selected for reporting a diagnostic result.

Step 1106 includes causing a diagnostic instrument to run the first test assay from the sample cartridge. In some embodiments, step 1106 includes running multiple test assays in the sample cartridge and storing multiple results from the test assays in a local memory of the diagnostic instrument. In some embodiments, step 1106 includes directing a diagnostic instrument to collect an image of the first test assay when completed and receiving the image of the first test assay from the diagnostic instrument.

Step 1108 includes transmitting, to the remote server, a first data set when the first test assay is completed.

Step 1108 includes receiving, from the remote server, a recommendation to a user of the diagnostic instrument based on the diagnostic result. In some embodiments, step 1108 includes receiving a request from the remote server to provide a test result from a second test assay.

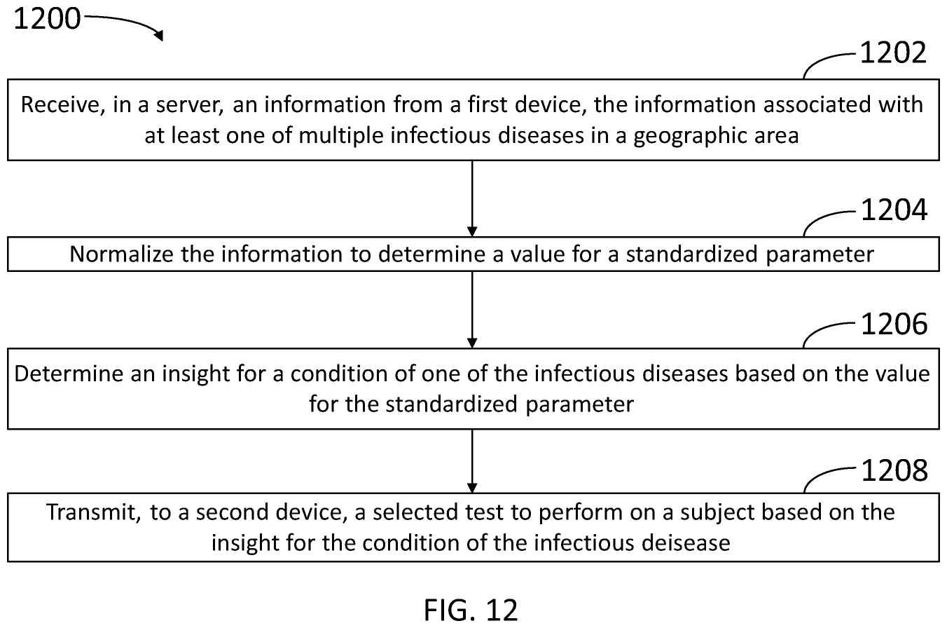

FIG. 12 is a flow chart illustrating steps in a method 1200 for collecting data to power an insight engine and decision support tool (e.g., insight engine 234 and clinical decision and support tool 240), according to some embodiments. Method 1200 may be performed at least partially by any one of the plurality of servers in collaboration with one or more client devices and databases, communicatively coupled through a network, as disclosed herein (cƒ. client devices 110 and 210, servers 130 and 230, databases 152 and 252, and networks 150 and 250). For example, at least some of the steps in method 1200 may be performed by one component in an architecture (cƒ. architectures 10 and 20), including a mobile device running code for a browser and an application to access a website for an insight engine that processes logic to evaluate and contextualize an infectious disease outbreak and a network management engine (e.g., insight engine 234 and network management engine 236). In some embodiments, the insight engine may include a clinical decision tool, a location tool, a statistics tool, and a consumer support tool, as disclosed herein (cƒ. clinical decision and support tool 240, location tool 242, and statistics tool 244). In some embodiments, one or more of the servers may also include an application layer to host and handle an application installed in a client device (e.g., application layer 215), so third-party users may access the disease analysis engine. Accordingly, at least some of the steps in method 1200 may be performed by a processor executing commands stored in a memory of one of the servers or client devices, or accessible by at least one of the servers or client devices (e.g., processors 212 and memories 220). Further, in some embodiments, at least some of the steps in method 1200 may be performed overlapping in time, almost simultaneously, or in a different order from the order illustrated in method 1200. Moreover, a method consistent with some embodiments disclosed herein may include at least one, but not all, of the steps in method 1200.

Step 1202 includes receiving, in a server, an information from a first device, the information associated with at least one of multiple infectious diseases in a geographic area. In some embodiments, step 1202 includes storing the information from the first device in a database. In some embodiments, step 1202 includes receiving, from the first device, data unrelated to the infectious disease, but significant to insights of decision support for one or more infectious diseases. For example, step 1202 may include receiving cell phone mobility data, or school attendance percentage by school or zip code, or allergen intensity data in a certain area, which data may be relevant to calculate probability and rate of infection for a population/subpopulation. In some embodiments, step 1202 may include receiving high resolution, patient specific information when requested. For example, step 1202 may include deriving determinants of health indicators such as socio-economic information, which may indicate that some individuals are more at risk of more severe outcomes for a certain type of disease.

Step 1204 includes normalizing the information to determine a value for a standardized parameter.

Step 1206 includes determining an insight for a condition (current or future) of one of the infectious diseases based on the value for the standardized parameter. In some embodiments, step 1206 also includes determining a clinical or operational decision support related to at least one of the infectious diseases. In some embodiments, step 1206 includes determining a probability that the disease will affect a certain aggregated portion of a population (resulting in best practice decision support). In some embodiments, step 1206 includes determining a probability and severity that the disease will affect a subject at the individual level (resulting in user specific decision support).

Step 1208 includes transmitting, to a second device, a selected test to perform on a subject based on the insight for the progression of the infectious disease. In some embodiments, step 1208 includes receiving a test result from the second device and updating the database with the test result. In some embodiments, step 1208 includes providing a firmware update to the second device in real time, or according to a pre-selected schedule. In some embodiments, step 1208 includes receiving, from the second device, an update of a preliminary test result, and providing to the second device a recommendation for a second test on the subject, based on the preliminary test result. In some embodiments, step 1208 includes receiving, from the second device, a negative test result for the subject, and transmitting a request for the second device to rerun the selected test until either a positive test result is obtained, or a probability of a true negative test result is higher than a pre-selected threshold. In some embodiments, step 1208 further includes transmitting the test result to a third-party server (e.g., an EHR, or a government database).

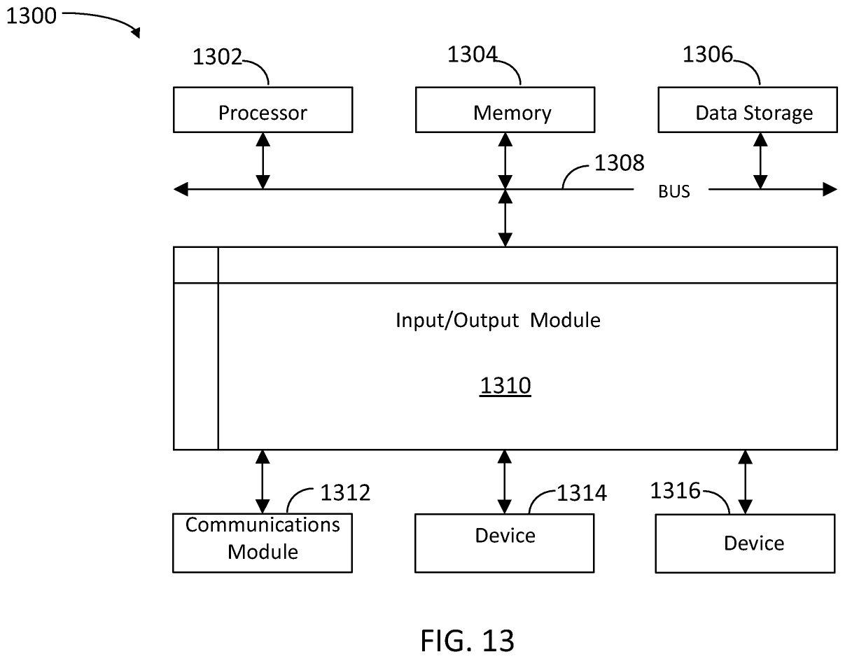

FIG. 13 is a block diagram illustrating an example computer system with which the client and network device of FIG. 1 and the methods of FIGS. 10-12 can be implemented. In certain aspects, computer system 1300 may be implemented using hardware or a combination of software and hardware, either in a dedicated network device, or integrated into another entity, or distributed across multiple entities.

Computer system 1300 (e.g., client devices 110 and 210, and servers 130 and 230) includes a bus 1308 or other communication mechanism for communicating information, and a processor 1302 coupled with bus 1308 for processing information. By way of example, the computer system 1300 may be implemented with one or more processors 1302. Processor 1302 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1300 can include, in addition to hardware, code that creates an execution environment for the computer program in question, e.g., code that constitutes processor firmware, a protocol stack, a database management system, an operating system, or a combination of one or more of them stored in an included memory 1304, such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 1308 for storing information and instructions to be executed by processor 1302. The processor 1302 and the memory 1304 can be supplemented by, or incorporated in, special purpose logic circuitry.

The instructions may be stored in the memory 1304 and implemented in one or more computer program consumer products, e.g., one or more modules of computer program instructions encoded on a computer-readable medium for execution by, or to control the operation of, the computer system 1300, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1304 may also be used for storing temporary variable or other intermediate information during execution of instructions to be executed by processor 1302.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1300 further includes a data storage device 1306 such as a magnetic disk or optical disk, coupled to bus 1308 for storing information and instructions. Computer system 1300 may be coupled via input/output module 1310 to various devices. Input/output module 1310 can be any input/output module. Exemplary input/output modules 1310 include data ports such as USB ports. The input/output module 1310 is configured to connect to a communications module 1312. Exemplary communications modules 1312 include networking interface cards, such as Ethernet cards and modems. In certain aspects, input/output module 1310 is configured to connect to a plurality of devices, such as an input device 1314 and/or an output device 1316. Exemplary input devices 1314 include a keyboard and a pointing device, e.g., a mouse or a trackball, by which a consumer can provide input to the computer system 1300. Other kinds of input devices 1314 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 1316 include display devices, such as an LCD (liquid crystal display) monitor, for displaying information to the user.

According to one aspect of the present disclosure, the client device 110 and servers 130 can be implemented using a computer system 1300 in response to processor 1302 executing one or more sequences of one or more instructions contained in memory 1304. Such instructions may be read into memory 1304 from another machine-readable medium, such as data storage device 1306. Execution of the sequences of instructions contained in main memory 1304 causes processor 1302 to perform the process steps described herein. One or more processors in a multi-processing arrangement may also be employed to execute the sequences of instructions contained in memory 1304. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back-end component, e.g., a data network device, or that includes a middleware component, e.g., an application network device, or that includes a front-end component, e.g., a client computer having a graphical consumer interface or a Web browser through which a consumer can interact with an implementation of the subject matter described in this specification, or any combination of one or more such back-end, middleware, or front-end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1300 can include clients and network devices. A client and network device are generally remote from each other and typically interact through a communication network. The relationship of client and network device arises by virtue of computer programs running on the respective computers and having a client-network device relationship to each other. Computer system 1300 can be, for example, and without limitation, a desktop computer, laptop computer, or tablet computer. Computer system 1300 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1302 for execution. Such a medium may take many forms, including, but not limited to, non-volatile media, volatile media, and transmission media. Non-volatile media include, for example, optical or magnetic disks, such as data storage device 1306. Volatile media include dynamic memory, such as memory 1304. Transmission media include coaxial cables, copper wire, and fiber optics, including the wires forming bus 1308. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

### Embodiments of the Present Disclosure Include

Embodiment I: A computer-implemented method includes receiving, from a diagnostic instrument, information regarding a sample cartridge to be used on a first subject, the information including a location data and a risk factor for one of multiple infectious diseases, the sample cartridge including multiple test assays for determining presence or absence of an analyte associated with a disease or disorder, such as an infectious disease, and/or diagnosing an infectious disease. The computer-implemented method also includes selecting, based on the location data and the risk factor, a test assay for reporting a diagnostic result, instructing the diagnostic instrument to run the test assay from the sample cartridge, receiving, from the diagnostic instrument, a first data set when the test assay is completed, and assessing a diagnostic result based on the first data set.

Embodiment II: A computer-implemented method includes providing, to a remote server, information regarding a sample cartridge to be used for a diagnostic test on a first subject, the information including a location data and a risk factor for at least one of a plurality of or multiple analytes of interest, such as an analyte indicative of presence or absence of an infectious disease, the sample cartridge including a plurality of or multiple test assays for determining presence or absence of an analyte associated with a disease or disorder, such as an infectious disease, and/or diagnosing an infectious disease. The computer-implemented method also includes receiving, from the remote server, based on the location data and the risk factor, a first test assay selected for reporting a diagnostic result, causing a diagnostic instrument to run the first test assay from the sample cartridge, and transmitting, to the remote server, a first data set when the first test assay is completed.

Embodiment III: A device includes a memory storing multiple instructions, a communications module configured to communicate with a remote server, and a processor configured to execute the instructions. Upon execution of the instructions, the processor causes the device to receive, from the remote server, an indication of a test assay to be selected for a subject, the test assay for determining presence or absence of an analyte associated with a disease or disorder, such as an infectious disease, and/or diagnosing an infectious disease, the indication based on at least one of a geolocated data indicative of a location of a diagnostic instrument and a prior diagnostic obtained with a one or more diagnostic instruments communicatively coupled with the remote server, to associate the test assay with multiple values to generate a data set diagnostic, the data set diagnostic stored within a memory of the diagnostic instrument, the multiple values related to one or more of: a test assay identifier, a test assay result, a patient identifier, and/or a diagnostic instrument identifier, and to transmit the data set diagnostic to the remote server for storage, wherein the remote server generates a report based on the data set diagnostic from each of the one or more diagnostic instruments, the report configured for transmission to a database housed on a database display on a second server or on an end-user workstation.

Embodiment IV: A computer-implemented method includes receiving, in a server, an information from a first device, the information associated with at least one of multiple infectious diseases in a geographic area, normalizing the information to determine a value for a standardized parameter, determining an insight for a condition of one of the infectious diseases based on the value for the standardized parameter, and transmitting, to a second device, a selected test to perform on a subject based on the insight for the condition of one of the infectious diseases.

Any one of embodiments I, II, III and IV may be combined with any one or more of the following elements, in any number or permutation.

Element 1, further including receiving, from a search engine, a datum associated with a search frequency for a selected keyword associated with an infectious disease in an area associated with the location data. Element 2, further including automatically updating the risk factor for the one of multiple infectious diseases based on a social network data. Element 3, further including providing, to the diagnostic instrument, an update for an application interface running the diagnostic instrument, based on the information regarding the sample cartridge and an identifier of the diagnostic instrument. Element 4, wherein selecting a test assay includes determining a false positive probability above a pre-selected threshold on the diagnostic result for the test assay. Element 5, further including preventing the diagnostic instrument to run a test assay based on the location data and the risk factor. Element 6, wherein selecting a test assay includes determining an infectious disease prevalence associated with the location data and the test assay. Element 7, further including communicating, to a client device associated with the location data, a request to run a test assay from a sample cartridge in the diagnostic instrument. Element 8, wherein assessing a diagnostic result includes retrieving, from a database, a second data set associated with a completed test assay for a second subject with a validated diagnostic result and comparing the first data set with the second data set. Element 9, further including providing a virtual assistant for the user based on the diagnostic result. Element 10, wherein transmitting a recommendation to the user includes providing a care pathway for the first subject when the diagnostic result is positive for an infectious disease.

Element 11, further including requesting, from the remote server, an epidemiology report for an infectious disease associated with the location data. Element 12, wherein running the first test assay from the sample cartridge includes running multiple test assays in the sample cartridge and storing multiple results from the test assays in a local memory of the diagnostic instrument. Element 13, wherein running the first test assay from the sample cartridge includes directing a diagnostic instrument to collect an image of the first test assay when completed and receiving the image of the first test assay from the diagnostic instrument. Element 14, wherein receiving a healthcare recommendation includes receiving a request from the remote server to provide a test result from a second test assay.

Element 15, further including providing the geolocated data. Element 16, wherein the communications module is configured to request, from the remote server, an epidemiologic report for a location associated with the geolocation data. Element 17, wherein the communications module is configured to request, from the remote server, a pre-test probability of a false positive result for the data set diagnostic. Element 18, wherein the communications module is configured to receive, from the remote server, a post-test communication alerting a user to conduct a higher performance diagnostic on the subject.

Element 19, further including storing the information in a database, receiving a test result from the second device; and updating the database with the test results. Element 20, further including updating a configuration of the second device in real-time. Element 21, further including updating a software command in the second device. Element 22, further including updating a firmware in the second device. Element 23, further including providing a firmware update to the second device at a pre-selected schedule or logic condition. Element 24, further including providing, to the second device, a probability of an outcome of the selected test for display in a graphic user interface of the second device. Element 25, further including receiving, from the second device, a query for a pre-test probability of an outcome prior to a completion of the selected test. Element 26, further including receiving, from the second device, an update of a preliminary test result, and providing to the second device a recommendation for a second test on the subject based on the preliminary test result. Element 27, further including receiving, from the second device, a negative test result for the subject, and transmitting a request for the second device to rerun the selected test until an outcome is obtained from one of: a positive test result, or a high confidence level of a negative test result. Element 28, further including receiving a test result from the second device and providing the test result to a third-party server. Element 29, further including transmitting a healthcare recommendation to a user of the diagnostic instrument based on the diagnostic result. Element 30, further including receiving, from the remote server, a healthcare recommendation based on a diagnostic result from the first data set.

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples, and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

1. A computer-implemented method, comprising:
receiving, from a diagnostic instrument, information regarding a sample cartridge to be used on a first subject, the information including a location data and a risk factor for one of multiple infectious diseases, the sample cartridge including multiple test assays for diagnosing the infectious diseases;
selecting, based on the location data and the risk factor, a test assay for reporting a diagnostic result;
instructing the diagnostic instrument to run the test assay from the sample cartridge;
receiving, from the diagnostic instrument, a first data set when the test assay is completed; and
assessing a diagnostic result based on the first data set.

2. The computer-implemented method of claim 1, further comprising receiving, from a search engine, a datum associated with a search frequency for a selected keyword associated with an infectious disease in an area associated with the location data.

3. The computer-implemented method of claim 1, further comprising automatically updating the risk factor for the one of multiple infectious diseases based on a social network data.

4. The computer-implemented method of claim 1, further comprising providing, to the diagnostic instrument, an update for an application interface running the diagnostic instrument, based on the information regarding the sample cartridge and an identifier of the diagnostic instrument.

5. The computer-implemented method of claim 1, wherein selecting a test assay includes determining a false positive probability above a pre-selected threshold on the diagnostic result for the test assay.

6. The computer-implemented method of claim 1, further comprising preventing the diagnostic instrument to run a test assay based on the location data and the risk factor.

7. The computer-implemented method of claim 1, wherein selecting a test assay includes determining an infectious disease prevalence associated with the location data and the test assay.

8. The computer-implemented method of claim 1, further comprising communicating, to a client device associated with the location data, a request to run a test assay from a sample cartridge in the diagnostic instrument.

9. The computer-implemented method of claim 1, wherein assessing a diagnostic result comprises retrieving, from a database, a second data set associated with a completed test assay for a second subject with a validated diagnostic result, and comparing the first data set with the second data set.

10. The computer-implemented method of claim 1, further comprising providing a virtual assistant for a user of the diagnostic instrument based on the diagnostic result.

11. The computer-implemented method of claim 1, further comprising transmitting a recommendation to a user of the diagnostic instrument, such as by providing a care pathway for the first subject when the diagnostic result is positive for an infectious disease.

12. A computer-implemented method, comprising:
providing, to a remote server, information regarding a sample cartridge to be used for a diagnostic test on a first subject, the information including a location data and a risk factor for at least one of multiple infectious diseases, the sample cartridge including multiple test assays for diagnosing the infectious diseases;
receiving, from the remote server, based on the location data and the risk factor, a first test assay selected for reporting a diagnostic result;
causing a diagnostic instrument to run the first test assay from the sample cartridge; and
transmitting, to the remote server, a first data set when the first test assay is completed.

13. The computer-implemented method of claim 12, further comprising requesting, from the remote server, an epidemiology report for an infectious disease associated with the location data.

14. The computer-implemented method of claim 12, wherein running the first test assay from the sample cartridge comprises running multiple test assays in the sample cartridge and storing multiple results from the test assays in a local memory of the diagnostic instrument.

15. The computer-implemented method of claim 12, wherein running the first test assay from the sample cartridge comprises directing a diagnostic instrument to collect an image of the first test assay when completed, and receiving the image of the first test assay from the diagnostic instrument.

16. The computer-implemented method of claim 12, further comprising receiving, from the remote server, a healthcare recommendation based on a diagnostic result from the first data set, and optionally receiving a request from the remote server to provide a test result from a second test assay.

17. (canceled)

18. A device, comprising:
a memory storing multiple instructions;
a communications module configured to communicate with a remote server; and
a processor configured to execute the instructions to cause the device to:
receive, from the remote server, an indication of a test assay for diagnosing an infectious disease to be selected for a subject, the indication based on at least one of a geolocated data indicative of a location of a diagnostic instrument and a prior diagnostic obtained with a one or more diagnostic instruments communicatively coupled with the remote server;
associate the test assay with multiple values to generate a data set diagnostic, the data set diagnostic stored within a memory of the diagnostic instrument, the multiple values related to one or more of: a test assay identifier, a test assay result, a patient identifier, and a diagnostic instrument identifier; and
transmit the data set diagnostic to the remote server for storage, wherein the remote server generates a report based on the data set diagnostic from each of the one or more diagnostic instruments, the report configured for transmission to a database housed on a database display on a second server or on an end-user workstation.

19. The device of claim 18, wherein execution of the instructions further cause the device to provide the geolocated data, and wherein the communications module is configured to request, from the remote server, an epidemiologic report for a location associated with the geolocation data.

20. (canceled)

21. The device of claim 18, wherein the communications module is configured to request, from the remote server, a pre-test probability of a false positive result for the data set diagnostic.

22. The device of claim 18, wherein the communications module is configured to receive, from the remote server, a post-test communication alerting a user to conduct a higher performance diagnostic on the subject.

23-33. (canceled)
